(19) United States
(12) Patent Application Publication
(10) Pub. No.: US 2024/0390562 A1
(43) Pub. Date: Nov. 28, 2024

(54) REMOVABLE FLANGE ADAPTERS FOR FLANGES USED IN SUCTION ACTIVATED SILICONE COLLECTION CUPS

(71) Applicant: Crystal NELSON, Redmond, OR (US)

(72) Inventor: Crystal NELSON, Redmond, OR (US)

(21) Appl. No.: 18/201,778

(22) Filed: May 25, 2023

(51) Int. Cl. A61M 1/06

(52) U.S. Cl. CPC A61M 1/0697; A61M 1/064; A61M 1/82

(57) ABSTRACT

This invention relates to a flange adapter for a flange used in a suction activated silicone collection cup. The flange adapter comprises a body defining a sidewall and an axial passageway. The body comprises a first end and a second end having a first diameter and a second diameter respectively, wherein the second diameter is greater than the first diameter. The body comprises at least one rib on its sidewall that enables it to be removably sealably engaged with an internal passage of the flexible flange. The second end has a frusto-conical shape. The flange adapter further comprises a removable section configured to be sealably engaged with the first end, wherein the removable extension comprises an axial passageway that coincides with the axial passageway of the flange adapter. The flange adapter and the removable section is made of a flexible polymeric material such as polyethylene, silicone, or combinations thereof.

### FIELD OF THE INVENTION

The present application relates to breast feeding of infants by their mothers. More particularly, it relates to an insert properly size the existing inner flange of a conventional portable suction activated silicone collection cup, to the determined size of the breast of a woman employing the suction activated silicone collection cup.

### BACKGROUND OF THE INVENTION

Breast feeding as is conventionally well known, is the natural process of feeding breast milk by the mother, to her infant. While this process can be accomplished in real time directly from mother to infant, frequently, especially with working mothers, it is accomplished by expressing milk from the breast of the mother and storing the milk for subsequent use where it may be employed in bottle-feeding of the expressed breast milk to the infant. Such milk expression is commonly accomplished by a suction activated silicone collection cup, also commonly known as breast pump, and in many cases portable breast pumps such as those manufactured and sold by Alexandria, VA-based Lansinoh Laboratories Inc., McHenry, IL-based Medela and Mequon, Wisconsin-based Haakaa.

Conventionally, such milk gathering and storing sessions for expressing breast milk employ such a suction activated silicone collection cup, which is configured with a flexible flange which is intended to sealably engage with the breast of the mother. A mechanical or other pump is operatively engaged to the flange which is placed in contact with one or both breasts of the user to thereby express milk. The expressed milk is routed by the pumping device to a container for storage and later use for feeding sessions.

When employing a breast pump to express milk it is an important consideration for both user comfort and maximizing the volume of expressed milk, to employ the flexible flange in proper contact with the breast. Such a conventional breast pump flange as provided with most conventional stationary and portable (user worn) breast pumps, should fit comfortably around the nipple of the breast of the user in a substantially sealed contact, prior to and during pumping by the engaged breast pump.

However, most manufacturers of such breast pumps, only provide a single flexible flange, which is intended to operatively and comfortably engage with the breast of the user. Such is frequently not the case and users can be subjected to discomfort as well as poor expression of milk from ill-fitting flanges which are not properly sized for comfortable and productive operative contact with the breast.

A comfortable and properly fitting flexible breast pump flange will insure that during use there is no discomfort and that an operative seal is accomplished with and to the breast of the user which provides for optimum suction to the breast and maximized milk expression. Further, such a comfortable and proper fit will also insure that when the mother later breast feeds the infant directly, that there is no tenderness or other discomfort.

The forgoing description concerning suction activated silicone collection cups or breast pumps employ for milk expression is intended to be illustrative and not exclusive. As such, no limitations on the invention described and claimed herein is to be associated with such. Further, additional limitations and features of the current art of breast pumps are well known, or such will become apparent to those skilled in the art upon a reading and understanding of the specification below and the accompanying drawings.

### SUMMARY OF THE INVENTION

The system and device herein in various modes provides for the adaptation of existing flexible flanges of suction activated silicone collection cups or breast pumps such as the Haakaa, Lansinoh and/or Medela and similar worn breast pumps, to a proper size for the actual breast size of the user. For example conventionally, a widely employed “standard” size for the opening of a flexible flange is formed to accommodate a breast nipple of a diameter which is substantially 36 mm. However, many women have nipple diameters which are smaller than the conventionally provided flexible flange.

The device herein is a solution to such poor fitting of a suction activated silicone collection cup flanges through the provision of one or a plurality of flange adapters. Each such flange adapter is formed of flexible polymeric material such as polyethylene or silicone to provide a soft comfortable operative contact against the breast of the user.

A first end or engagement end of each flange adapter has an exterior circumference adapted to form a sealed engagement with the internal circumference of the passage of the existing breast pump flange. This insures that any suction communicated to the engaged end of the existing breast pump flange, will be fully communicated to the internal passage of the flange adapter herein.

A second end of the flange adapter herein is formed with a larger diameter than the first end in a frusto-conical shape where the wider end is positionable to form a seal on the breast of the user in the area of the breast surrounding the nipple.

An axial passage communicates through the flange adapter from the first end where the axial passage is placed in a sealed communication with the suction communicated to the flexible flange provided with and mounted to the suction activated silicone collection cup. It is the diameter of this axial passage, in each of the flexible adapter flanges provided herein, which is sized to comfortably engage with the nipple of the user.

Thus, when the device herein is provided as a kit of available flange adapters, each will have the same exterior diameter which the circumferential surface sized to sealably engage with the existing internal passage of the flange of the suction activated silicone collection cup. However, each of the axial passages of each of the multiple or plurality of available adapter flanges, is formed with a different diameter of the internal axial passage. Currently, such internal axial passages are provided in diameters from 13 mm to 19 mm in increments increasing by 2 mm from the smallest to the largest diameter. However, more sizes can be provided in for example increasing increments of 1 mm from 13 mm to 19 mm or more. To render it easy to pick the proper sized flexible flange adapter herein for the user, the actual size of the internal axial passage is embossed into the flexible polymeric material forming the flange adapter. This makes it easy for the user to employ existing light to show the size of each flange adapter.

Additionally provided at the first end of some of the flange adapters, is an opening formed through the sidewall of the flange adapter. In some brands of portable suction activated silicone collection cups, the suction communicated to the provided flange, is communicated to the internal sidewall. While such a configuration will work where there is no flange adapter inserted, in such a configured suction activated silicone collection cup, an opening through the sidewall to the axial passage is required to communicate the suction to the breast of the user when operatively engaged with the flange adapter herein.

This opening can be provided in the form of a removable portion of the sidewall of the flange adapter herein. This will allow the flange adapter to be operatively engaged with the suction activated silicone collection cups, which communicate suction to an opening in the existing flange which aligns with the axial passage of the flange adapter herein at the first end thereof. Should however it be determined that the suction passage does not align and is communicated through the sidewall of the existing flange, then the user has the option of removing the portion of the sidewall to form the opening therein. The removable portion can rendered frangible by forming a cut in the shape of the removable portion, which does not complexly sever the sidewall. It would thus remain sealed until removed which would be done by cutting the remaining material or tearing it.

An additional preferred mode of the device, would also provide removable end portions. This is because different brands of suction activated silicone collection cups can have length differences with regard to the internal passage of the existing breast flange. Some require a longer first end on the flange adapter herein, to operatively seal with the existing internal passage and some may require a shorter first end. As such, by rendering portions of the first end of the flange adapter herein, easily removable in the same fashion as the removable openings, the user will be able to adapt the flange adapter herein, to the length of the internal passage of any existing flange of existing breast pumps.

With regard to the above disclosure, those skilled in the art will appreciate that the conception upon which this disclosure is based may readily be utilized as a basis for designing other breast flange sizing systems and for carrying out the several purposes of the present disclosed device. It is important, therefore, that the claims be regarded as including such equivalent construction and methodology insofar as they do not depart from the spirit and scope of the present invention.

As used in the claims to describe the various inventive aspects and embodiments, “comprising” means including, but not limited to, whatever follows the word “comprising”. Thus, use of the term “comprising” indicates that the listed elements are required or mandatory, but that other elements are optional and may or may not be present. By “consisting of” is meant including, and limited to, whatever follows the phrase “consisting of”. Thus, the phrase “consisting of” indicates that the listed elements are required or mandatory, and that no other elements may be present. By “consisting essentially of” is meant including any elements listed after the phrase, and limited to other elements that do not interfere with or contribute to the activity or action specified in the disclosure for the listed elements.

Thus, the phrase “consisting essentially of” indicates that the listed elements are required or mandatory, but that other elements are optional and may or may not be present depending upon whether or not they affect the activity or action of the listed elements. The term “substantially” when employed herein, means plus or minus twenty percent unless otherwise designated in range.

It is an object of the present invention to provide flange adapters which will allow for alteration of the diameter of the existing internal passage of flanges of suction activated silicone collection cup to one matching the diameter of the nipple of the user.

These and other objects, features, and advantages of the presently disclosed breast pump flange adapters system herein as well as the advantages thereof over existing prior art, which will become apparent from the description to follow, are accomplished by the improvements described in this specification and hereinafter described in the following detailed description which fully discloses the invention, but should not be considered as placing limitations thereon.

### BRIEF DESCRIPTION OF DRAWING FIGURES

The accompanying drawings, which are incorporated herein and form a part of the specification, illustrate some, but not the only or exclusive examples of embodiments and/or features of the disclosed flange adapter of suction activated silicone collection cups. It is intended that the embodiments and figures disclosed herein are to be considered illustrative of the invention herein, rather than limiting in any fashion.

In the drawings:

FIG. 1 shows an existing portable suction activated silicone collection cup and the conventional flexible flange provide therewith for engagement to the breast of the user, and FIG. 1A shows an example of the flange adapter herein positioned for insertion into the internal passage of the existing flange.

FIG. 2 shows the flange adapter herein having an exterior diameter which sizes the circumference at the first end of the flange adapter to form a seal with the interior circumference of the wall defining the internal passage of the existing flange.

FIG. 3 depicts a flange adapter herein having an axial passage sized to comfortably and operatively engage with the nipple of a user having a 15 mm diameter and having the exterior circumference similar in size to that of FIG. 1.

FIGS. 4-5 show other configurations of the flange adapter 20 herein, wherein the exterior circumference is similar to that of the device in FIG. 1, but the diameter of the axial passage of the flange adapters varies between 17 mm to 19 mm.

FIG. 6 depicts the flange adapter herein, having a removable section in the sidewall at the first end thereof.

FIG. 7 shows the flange adapter herein in a mode with removable sections at the first end thereof to thereby adjust the length and showing removable sections in each removable portion which can be separated if necessary to accommodate side-positioned suction in the internal passage of the existing flange.

FIG. 8 shows the first end of the flange adapter herein and examples of preferred dimensions thereof and also shows the opening formed in the sidewall which would be positioned to communicate suction to the axial passage of the flange adapter herein.

FIG. 9 is a side view of an exemplary removable nipple.

FIG. 10 is a top isometric view of the removable nipple of the invention.

FIG. 11 is a bottom isometric view of the removable nipple.

FIG. 12 is a top view of the removable nipple.

FIG. 13 is a bottom view of the removable nipple.

FIG. 14A is a sectional view of one exemplary embodiment of the removable nipple of the invention.

FIG. 14B is a sectional view of another exemplary embodiment of the removable nipple.

FIG. 15 is an exemplary side view of a suction activated silicone collection cup comprising the flange adapter of the invention.

### DETAILED DESCRIPTION OF THE INVENTION

In this description, the directional prepositions of up, upwardly, down, downwardly, front, back, top, upper, bottom, lower, left, right, front, back, and other such terms refer to the device as it is oriented and appears in the drawings and are used for convenience only and such are not intended to be limiting in any fashion, or to imply that the device has to be used or positioned in any particular orientation.

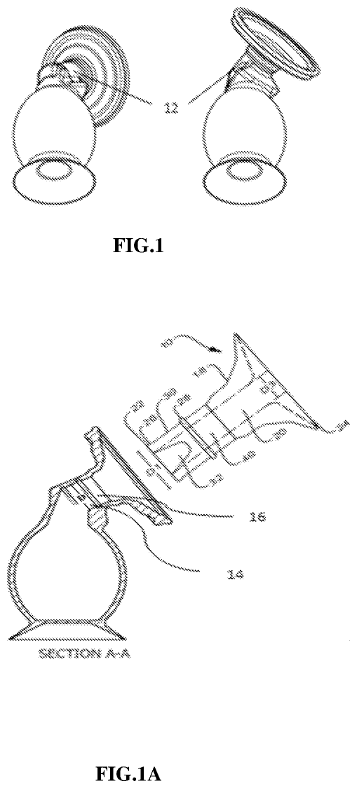

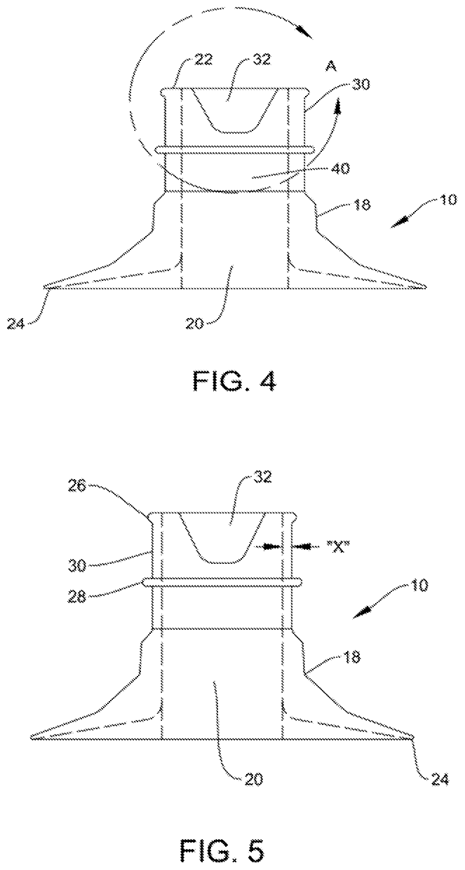

Now referring to drawings in FIGS. 1-9 wherein similar components are identified by like reference numerals, there is seen in FIG. 1 an example of a conventional existing portable suction activated silicone collection cup 12, interchangeably referred to as breast pump or collection cup, which is sold for both portable use where it is enclosed in a nursing bra and engaged to the breast of the user, as well as modes of such collection cups 12 which are counter-positioned or otherwise used at home.

Such breast pumps 12 conventionally have a flexible flange 14 which is provide to the user with the breast pump 12 which is intended for direct operative engagement with the breast of the nursing mother. As noted, such conventional flexible flanges 14, are provided with an internal passage 16 which is intended to be a one size fits all configuration where the diameter “D” of the internal passage 16, is intended to operatively engage with the nipple of the breast of the nursing mother.

As noted, this single sized diameter “D” of the conventional flexible flange 14, while easing the requirements of the manufacturer, does not always serve the user well. This is because the diameter and thus the circumference of the nipple of the breast of nursing mothers, can vary widely. Thus, the provision of the flexible flange 14, with a single diameter “D” yielding a single size to the internal circumference of the internal passage 16 may not provide a comfortable and operative fit, to many users who may have smaller diameter nipple sizes than that of the diameter “D” of the internal passage 16 of the provided flexible flange 14.

The flange adapting device 10 and system herein, when employed in combination with a conventional portable or in home breast pump 12 allows the user to adjust the diameter “D” of the internal passage 14 of the conventional flexible flange 14, to one with is both more comfortable. The user such as a nursing mother, will choose and employ a flange adapter device 10 formed with a body 18 which has a diameter D1 yielding an exterior circumference which removably sealably engages with the internal passage 16. This removably sealed engagement allows the user to remove the flange adapter device 10 as needed for both cleaning, as well as the ability to choose a different flange adapter device 10 with a different sized axial passage should their needs change as to the size of their nipple on any given day.

In use, the device 10 may be sold in varying sizes, or a kit including multiple flange adapter devices 10 can be provided. The user will choose a flange adapter having a body 18, which has the diameter D1 which will form an external circumference configured for a sealed removable engagement within the internal passage 16 of the provided flexible flange 14 of the breast pump 12.

Once a flange adapter device 10 having a body 18 with the diameter D3 determined to be both a comfortable and operable fit to the nipple of the user has been chosen from the plurality thereof available, it will be slid into removable sealed engagement with the internal passage 16 of the conventional flexible flange 14. Thereafter, the user will slide the axial passage 20 sized for the diameter and circumference of the nipple of their breast, to a removable engagement thereon in an as-used positioning. With the body 18 of the chosen flange adapter device 10 in this as-used positioning, the suction communicated to the internal passage 16 of the flexible flange 14, is communicated through the axial passage 20 whereupon milk is expressed and drawn into a reservoir of the breast pump 12.

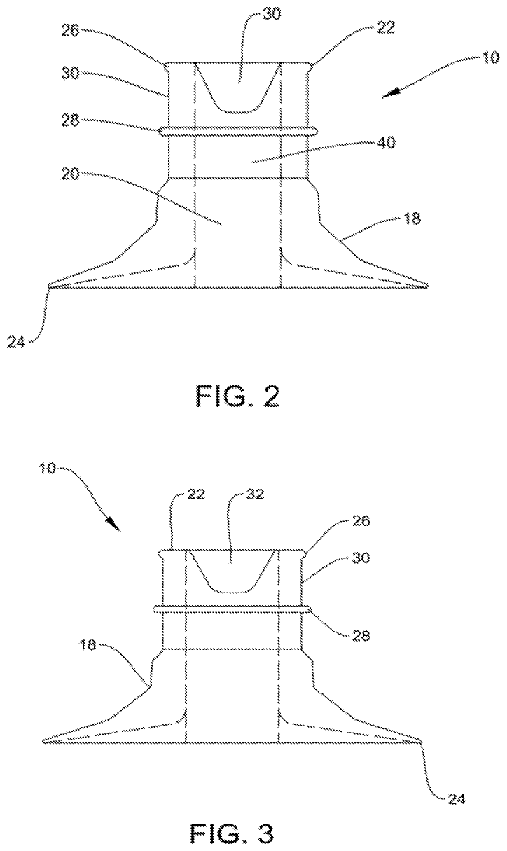

As shown in FIGS. 2-5, the device 10 herein is provided such that the user may choose from a plurality of such devices 10 where each has a body 18, configured at a first end 22 having a diameter yielding an exterior circumference at the first end 22 which is configured to sealably engage with the internal passage 16 of the flexible flange 14. The body 18 may be configured to engage with the internal passage 16 of flexible flanges 14 of the breast pumps 12 of a single manufacturer where needed, or the pumps 12 sold by multiple different manufacturers.

At the first end of the device 10 herein, is shown a first sealing ridge 26 and a second sealing ridge 28 both extending in the form of an annular projecting ring from the exterior circumferential surface of the body 18 of the device 10 herein adjacent the first end 2 thereof. This forms an annular channel 30 between the first sealing ridge 16 and second scaling ridge 28. For breast pumps 12 having a side-positioned suction component, a suction opening 32 may be provided which will communicate suction from the breast pump 12 through this annular channel 30 to the axial passage 20 and to the breast.

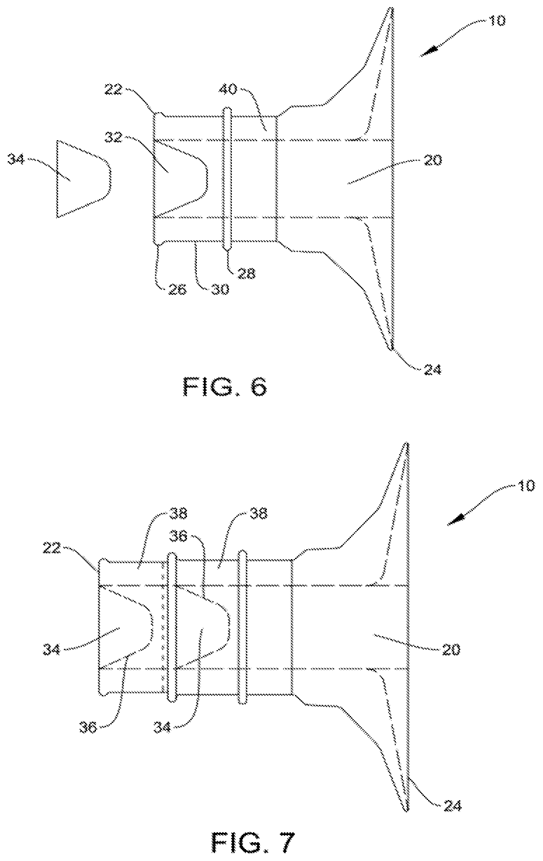

In modes of the device 10 engaged with a breast pump 12 providing suction to an opening aligned with the axis of the internal passage 16, the suction opening 32 may not be required. Consequently, the device 10 can be provided as shown in FIGS. 6-7, with a removable section 34 which forms a sealed sidewall of the body 18 at the first end 22 when in place. However, along the perimeter edge of the removable section 34 the sidewall may be cut partially with a formed recess 36 (FIG. 7) which will render the removable section 34 frangible such that it may be torn from the sidewall to form the suction opening 32 if needed. If not needed, the removable section 34 would remain in place thereby forming a sealed sidewall and suction would be communicated from the breast pump 12 through a passage aligned with the internal passage 16.

Shown in FIG. 7, the flange adapter device 10 herein, may also be provided in a configuration with removable end sections 38 positioned at the first end 22. Or a plurality of end sections 38 may be positioned in sequence starting at the first end 22. As shown, the removable sections 34 may also be provided with each such removable end section 38.

The removable end sections 38 if provided will allow for an adjustment of the length of the body 18 of the device 10 to accommodate the differing length of the internal passages 16 of conventional flexible flanges 14 of different breast pump 12 manufacturers. Where needed a shortening of the body 18 of the device 10 herein may be accomplished by the removal of one or more removable end sections 38. These removable end sections 38 if provided will be rendered frangible in the same fashion as the removable sections 34 which may be torn from the body 18 to form the suction openings 32 such as be a cut forming a recess 36 into the external surface of the body 18.

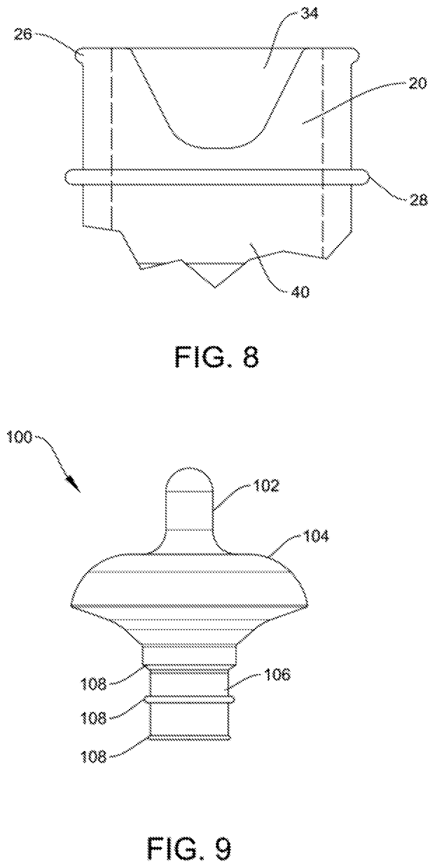

In FIG. 8 is shown an enlargement of the first end of the body 18 forming the flange adapter device 10 herein. Shown are examples of the dimensions of current versions thereof. However those skilled in the art will realize that such dimensions may change depending on the manufacturer of the breast pump 12 and the flexible flange 14 provided therewith.

Table given below provides exemplary minimum wall thicknesses associated with various exemplary part sizes of the removable flange of the invention which may be formed to yield varying diameters D3 of the axial passage 20.

MINIMUM WALL THICKNESS

| PART SIZE | “X” DIMENSION |
|---|---|
| 13 | 4.25 |
| 15 | 3.25 |
| 17 | 2.25 |
| 19 | 1.25 |

However, the external dimensions at the first end 26 of the body 18 may remain static or the same. Maintaining the exterior of the body 18 at the first end 22 thereof thereby configures the first end 22 of each member of a kit having a plurality of flange adapter devices 10 therein, removably sealably engageable with the internal passage 16 of the intended flexible flange 14. Varying the sidewall thickness however will vary the diameter D3 of the axial passage 30 thereby allowing for multiple sizes where the user may choose the body 18 which has the diameter D3 of the axial passage 20 which fits their nipple comfortably and allows for communication of suction from the breast pump 12 to express and store breast milk.

Finally, in a method of employment of the device 10 herein depicted in FIGS. 1-8, the user will first determine a diameter of the nipple of their breast. Such may be determined using rulers or calipers or other available diameter measuring devices. Once a diameter of the nipple is determined, the user will choose the device 10 having the appropriate axial passage 20 diameter D3 that most closely matches their nipple diameter. Indicia 40 identifying the diameter D3 of the axial passage 20 of the body 18 of each individual device 10 available allows the user to choose a matching body 18.

Once the body 18 is chosen from a plurality thereof having differing axial passage 20 diameters, the first end of the chosen body 18 will be engaged with the internal passage 16 of the flexible flange 14 of the breast pump 12 manufacturer. Thereafter the breast pump 12 would be energized and suction will be communicated to the nipple of a breast in operative engagement at the second end 24 of the chosen body 18 to express milk.

Where the user determines that suction communicated to the internal passage 16 of the flexible flange 14 is from a side-positioned source, if a suction opening 32 is already positioned on the chosen body 18 it will be aligned with the suction conduit of the flexible flange 14. If the suction opening is not present yet, the user will tear the removable section 34 from the sidewall of the body 18, and then position the formed suction opening 32 aligned with the side-positioned suction conduit. Thereafter, the breast pump 12 is energized.

In some embodiments, the invention may further include a removable nipple that can be attached to the removable flange. FIG. 9 is a side view of an exemplary removable nipple depicted by numeral 100. The removable nipple may be configured to be fitted into the removable flange in a facile manner such that it enables conversion of the breast pump to be configured for feeding to a baby. In some embodiments, the removable nipple may be made of the same material as the removable flange, such as a silicone elastomer, more specifically an FDA approved BPA-free silicone elastomer. The removable nipple 100 comprises a feeding portion 102 that is configured to be received by the lips of a baby, a flat portion 104 against which a baby's lips would rest while feeding, and a neck portion 106 that is used to connect to the removable flange. The neck portion may comprise one or more ribs 108 that are configured to be received by the removable flange and remain in place.

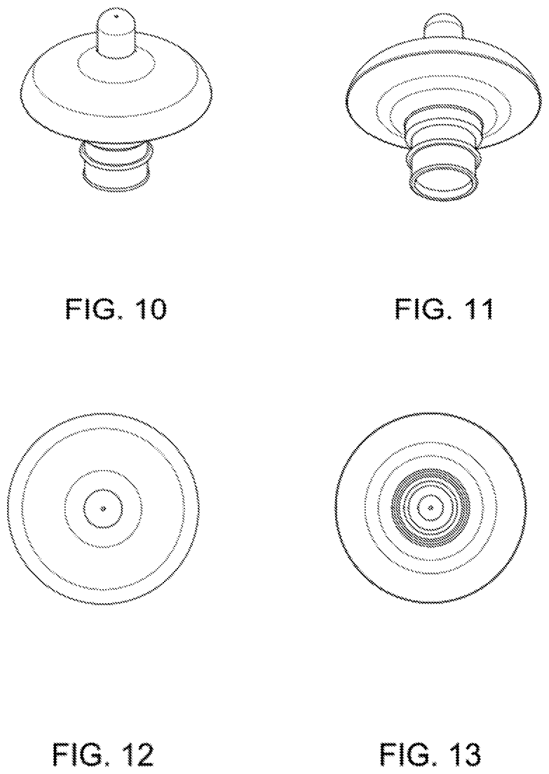

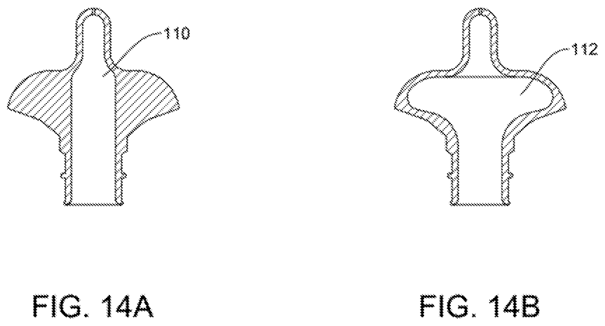

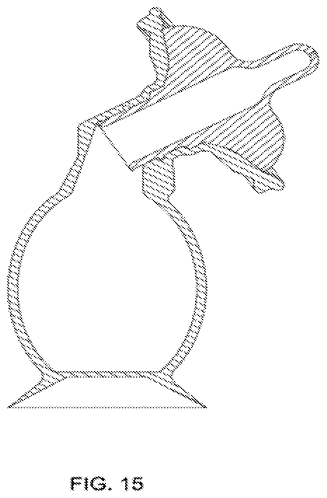

FIG. 10 is a top isometric view of the removable nipple of the invention, and FIG. 11 is a bottom isometric view of the removable nipple. FIG. 12 is a top view and FIG. 13 is a bottom view of the removable nipple. FIG. 14A is a sectional view of one exemplary embodiment of the removable nipple of the invention, and FIG. 14B is a sectional view of another exemplary embodiment of the removable nipple. In FIG. 14A, a narrower inner flow path 110 is defined as opposed to that in FIG. 14B in which a broader flow path 112 is defined therein. Other such variations for the flow path will become apparent to one skilled in the art, and is encompassed to be within the scope of the invention. FIG. 15 is an exemplary side view of a breast pump comprising the flange adapter of the invention that further comprises the removable nipple, such that it is configured to be used to feed a baby.

Thus, it can be seen that the attachments of the invention facilitates the adaptation of any breast pump to any breast size and/or nipple size with considerable ease, and further makes it configurable for feeding the collected milk to a baby.

It should be noted that any of the different depicted and described configurations and components of the breast flange adapter device herein can be employed with any other configuration or component shown and described as part of the device herein. Additionally, while the present invention has been described herein with reference to particular embodiments thereof and/or steps in the method of production or use, a latitude of modifications, various changes and substitutions are intended in the foregoing disclosure, and it will be appreciated that in some instance some features, or configurations, of the invention could be employed without a corresponding use of other features without departing from the scope of the invention as set forth in the following claims. All such changes, alternations and modifications as would occur to those skilled in the art are considered to be within the scope of this invention as broadly defined in the appended claims.

Further, the purpose of any abstract of this specification is to enable the U.S. Patent and Trademark Office, the public generally, and especially the scientists, engineers, and practitioners in the art who are not familiar with patent or legal terms or phraseology, to determine quickly from a cursory inspection the nature and essence of the technical disclosure of the application. Any such abstract is neither intended to define the invention of the application, which is measured by the claims, nor is it intended to be limiting, as to the scope of the invention in any way.

1. A flange adapter for a flange of a suction activated silicone collection cup, the flange adapter comprising:
a body defining a sidewall and an axial passageway, the body comprising a first end and a second end having a first diameter and a second diameter respectively, wherein the second diameter is greater than the first diameter; wherein the second end is configured to be sealably engaged with a breast of a user;
the body comprising at least one rib on its sidewall that enables it to be removably sealably engaged with an internal passage of the flexible flange.

2. The flange adapter of claim 1, wherein the body comprises two ribs, wherein the first rib is positioned at the first end, and the second rib is positioned between the first end and the second end.

3. The flange adapter of claim 1 further comprising a removable section configured to be sealably engaged with the first end, wherein the removable extension comprises an axial passageway that coincides with the axial passageway of the flange adapter.

4. The flange adapter of claim 3, wherein the removable section comprises at least one recess.

5. The flange adapter of claim 1, wherein the flange adapter is made of a flexible polymeric material.

6. The flange adapter of claim 3, wherein the removable section is made of a flexible polymeric material.

7. The flange adapter of claim 5, wherein the flexible polymeric material is selected from a group comprising polyethylene, silicone, or combinations thereof.

8. The flange adapter of claim 1, wherein the second diameter ranges from about 13 millimeters to about 19 millimeters.

9. The flange adapter of claim 1, wherein a thickness of the sidewall varies along a length of the axial passageway.

10. The flange adapter of claim 1, wherein the second end has a frusto-conical shape.

11. The flange adapter of claim 1, further comprising a removable nipple.

12. A suction activated silicone collection cup comprising the flange adapter of claim 1.

13. The suction activated silicone collection cup of claim 12 further comprising a removable nipple.
